(19) United States
(12) Patent Application Publication
(10) Pub. No.: US 2019/0194587 A1
(43) Pub. Date: Jun. 27, 2019

(54) FLOATING HORIZONTAL TUBULAR PHOTOBIOREACTOR SYSTEM WITH INTEGRATED MANIFOLDS FOR HOUSING PUMPING AND PROCESS MONITORING AND CONTROL DEVICES

(71) Applicants: Michael Welch, Tampa, FL (US); Lawrence Walmsley, Coral Gables, FL (US); Andreas Meiser, Saarbrucken (DE); George Philippidis, Tampa, FL (US)

(72) Inventors: Michael Welch, Tampa, FL (US); Lawrence Walmsley, Coral Gables, FL (US); Andreas Meiser, Saarbrucken (DE); George Philippidis, Tampa, FL (US)

(21) Appl. No.: 16/229,154

(22) Filed: Dec. 21, 2018

Related U.S. Application Data
(60) Provisional application No. 62/609,738, filed on Dec. 22, 2017.

(51) Int. Cl. C12M 1/00; C12M 1/12; C12M 1/09; C12M 1/06; C12P 13/04; C12P 7/06; C12P 5/02; C12P 3/00; C12P 7/64; C12P 21/00

(52) U.S. Cl. CPC C12M 21/02; C12P 21/00; C12M 21/12; C12M 23/56; C12M 23/26; C12M 29/20; C12M 29/00; C12M 27/02; C12M 41/06; C12P 13/04; C12P 7/065; C12P 5/023; C12P 3/00; C12P 7/6409; C12M 23/06

(57) ABSTRACT

This invention relates to a floating horizontal tubular photobioreactor system comprising a photosynthetically active area made of flexible tubes connected to rigid manifolds with an integrated system for mixing, aeration and degassing. This invention further relates to methods of using the floating photobioreactor system.

### FIELD OF THE INVENTION

This invention relates to a floating horizontal tubular photobioreactor system comprising a photosynthetically active area made of tubes from plastic film integrated with manifolds at the ends which enable the system to float and house all auxiliary equipment required for pumping, aerating, degassing, and process monitoring and control. This invention further relates to methods of using the floating photobioreactor system.

### BACKGROUND OF THE INVENTION

The majority of today's photosynthetic algae production is practiced in open systems (“open ponds”). These open systems are susceptible to water evaporation and contamination by foreign algae, protozoa, parasites or other microorganisms. Thus, only a very limited number of algae with very specific growth characteristics can be cultivated in open systems. For example, the species Dunaliella grows in extremely salty water, in which hardly any other organisms can grow, and as such, it can be cultivated in an open system. In addition to being susceptible to contamination, open systems demonstrate low productivity. This results in a high production cost of algae. They also achieve usually only low biomass densities which results in high cost for pumping/treating the required water, harvesting and downstream processing. Depending on the area of application, the production costs may be too high and not economical. For example, the production costs of algae for use in the energy sector are too high to be profitable.

As an alternative to open systems, a large number of closed systems (“photobioreactors”) have been developed. These include horizontal, flat photobioreactors, tubular photobioreactors and vertical, flat photobioreactors. Many of these photobioreactors are less susceptible to contamination and can reach higher productivities than open systems. However, photobioreactors have investment costs that are too high for many applications to achieve economical production of algae cell mass.

One example of a horizontal photobioreactor is a horizontal film reactor which can float on a pool of water. See, e.g., U.S. Pat. No. 8,859,262 B2 and WO 2008/079724. In general, these horizontal film reactors have low investment cost, low susceptibility to contamination and good productivities. The low investment cost is due, in part, to the fact that these reactors can be manufactured using a low cost plastic film, e.g., polyethylene (“PE”). PE films can be processed easily by heat welding. See, e.g., WO 2008/079724. The resulting structure is flexible, which can facilitate the mixing of the system.

While the flexibility of the horizontal reactor gives rise to a number of advantages, it also creates some major challenges that prohibit commercial use.

A major challenge is keeping a steady flow of water in the reactor to ensure adequate mixing. The constant movement of the algae solution is required to avoid settling of the algae cells to the bottom of the reactor that diminishes photosynthesis, to avoid thermal stratification and to provide mixing for sufficient nutrient access by the algae.

When such a flexible film photobioreactor is floating on a body of water, the pressure required to create flow inside the flexible photobioreactor generates a downward force that forces the bottom sheet of the flexible film photobioreactor to sink into the body of water. This distorts the cross-sectional shape of the flow, leading to energy dissipation and and flow failure due to insufficient flow velocity.

In general, it will be possible to reduce or eliminate this distortion by connecting the top and bottom sheets in various ways. 1. One way is by sealing together the top and bottom sheets in such as a way as to create channels through which the flow must move e.g., U.S. Pat. No. 9,376,656 B2. 2. Another solution is to shape the photosynthetically active area (through which the algae culture flows) as narrow parallel tubes instead of one large plastic sheet. These tubes would be located in parallel proximity to each other to create the required surface area. The pressure of the flow will keep the tubes open but due to their circular shape they cannot deform further. The pressure in fact will assist in keeping the culture in motion.

Since the photosynthetically active area is comprised of flexible material that easily sinks, the system components for mixing, aerating and degassing have to be located away from the photosynthetically active area.

In terms of mixing, there are two major methods used: 1. Use of axial flow using a positive displacement pump; and 2. Airlift pump or any other way to blow air or any gas mixture into the culture liquid to create a liquid movement.

In addition to mixing, aeration is another challenge in a flexible reactor. Good aeration is crucial to achieving high productivity as CO₂ is the carbon source required for the growth of algae and the air removes oxygen produced by the algae to prevent its deleterious accumulation in the culture. In general, three major means of aeration exist: 1) airlift pumps; 2) internal bubbling (bubbling of air into the reactor at one or several locations); and 3) semi-permeable diaphragm.

### SUMMARY OF THE INVENTION

The present invention introduces two rigid manifolds into the floating horizontal tubular photobioreactor system as a technology enabler that helps provide adequate mixing and aeration to the algae culture, helps the system float, and houses within the system all the essential peripheral equipment, including pumps, aeration and degassing areas, and process monitoring and control sensors and probes. The rigid manifolds provide an integrated solution for mixing, aeration, degassing, and process monitoring and control. In addition, the rigid manifold's height out of the water can be adjusted to increase pressure inside the tubes to the appropriate level.

The present invention describes a cooling system to remove heat from inside the photobioreactor via the use of an integrated heat exchanged constructed of the same flexible material and attached directly to the photosynthetically active area.

The present invention describes a system and process to autonomously estimate the density of the algae culture inside the photobioreactor system by the use of light reflection.

The present invention also provides methods of using the floating photobioreactor system. The floating photobioreactor system may be used to grow photosynthetic or mixotrophic organisms. Examples for photosynthetic organisms could be microalgae, macroalgae, cyanobacteria, other photosynthetic active bacteria or even higher plants, such as duckweed. Alternatively, the floating photobioreactor system may be used to produce a biomass, a biofuel or a product selected from biochemicals, amino acids, fine chemicals, nutriceuticals, pharmaceuticals, energy products, protein, feed for cattle, fish and other species, protein source for human nutrition and mineral rich food for human consumption.

### BRIEF DESCRIPTION OF THE FIGURES

FIG. 1 is a three-dimensional view of a floating tubular photobioreactor system with two rigid manifolds integrated on the ends

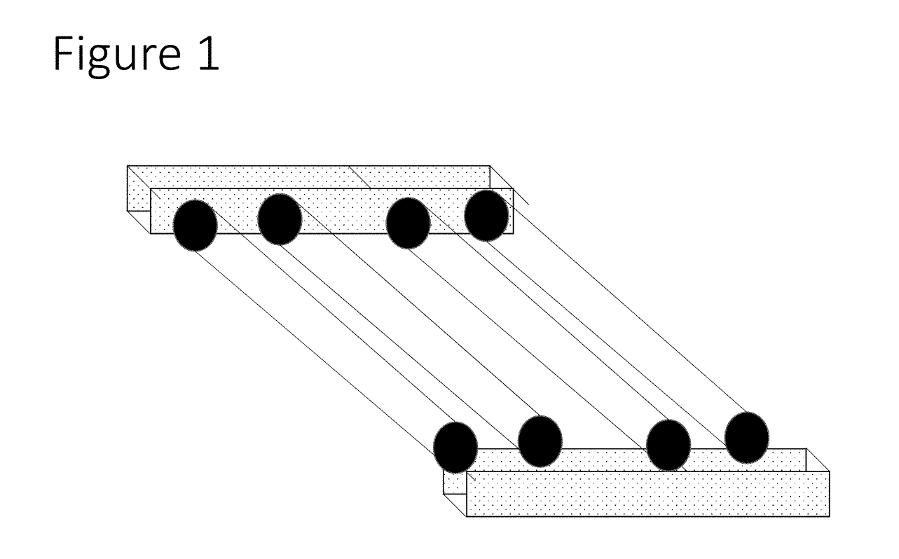

FIG. 2 is a cross section of a cutout of a manifold with integrated stirring, aeration, degassing, and process monitoring and control sensors and probes

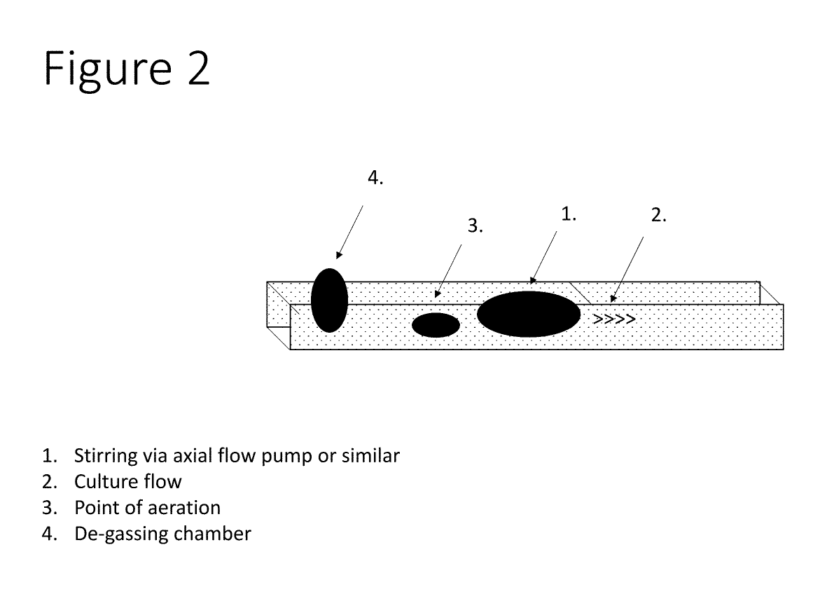

FIG. 3 is a cross section of a manifold illustrating the flow of liquid through the de-gassing chamber

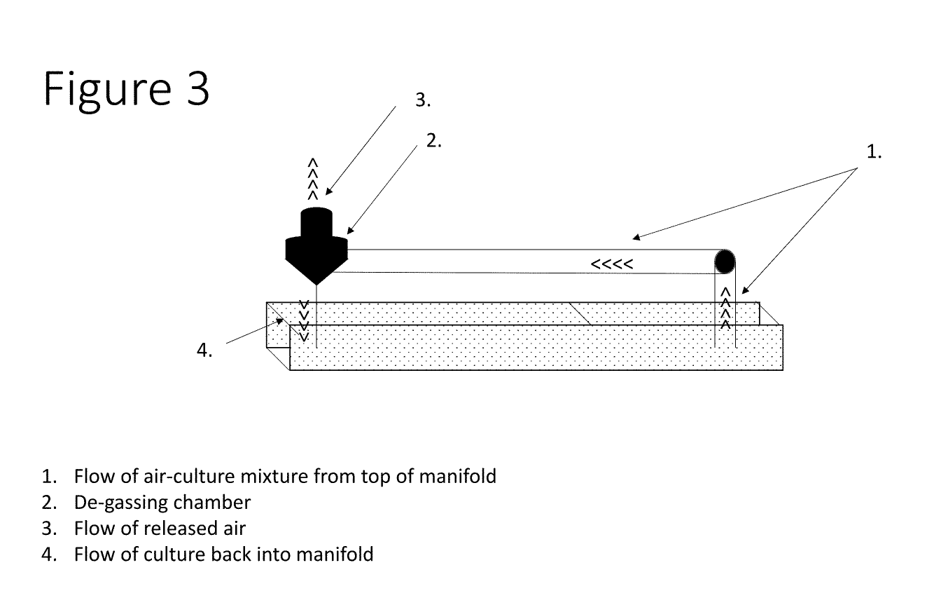

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

The term “a” or “an” may mean more than one of an item.

The terms “and” and “or” may refer to either the conjunctive or disjunctive and mean “and/or”.

The term “about” means within plus or minus 10% of a stated value. For example, “about 100” would refer to any number between 90 and 110.

The term “aeration” includes the addition of CO₂ and the removal of oxygen at the same time, even if not both functions are always mentioned explicitly.

The present invention provides a floating horizontal tubular photobioreactor system comprised of a photosynthetically active area made of flexible tubes connected to two rigid manifolds on the ends with an integrated system for mixing, aeration and degassing.

The present invention introduces two rigid manifolds into the floating horizontal tubular photobioreactor system as a technology enabler that helps provide adequate mixing and aeration to the algae culture, helps the system float, and houses within the system all the essential peripheral equipment, including pumps, aeration and degassing areas, and process monitoring and control sensors and probes. The rigid manifolds provide an integrated solution for mixing, aeration, degassing, and process monitoring and control. In addition, the rigid manifold's height out of the water can be adjusted to increase pressure inside the tubes to the appropriate level.

The advantages of integrating all functions into rigid manifolds at the ends of the floating, flexible reactor are multiple:
The integrated pump provides an energy-efficient means of mixing the media in the floating low cost reactor.
The integrated pump represents a means to achieve a good mass transfer between gas and liquid phase, especially for CO₂ and oxygen.
By integrating the stirring mechanism into the reactor fewer parts will be required, thus lowering the capital and maintenance costs.
By integrating the manifolds into the reactor the algae suspension does not need to be transported out of and into the reactor, thus saving energy and cost.
The manifolds are built from a rigid material can serve as an anchor to fix the position of the floating flexible reactor.

The benefits of the algae running through the tubes which make up the photobioreactor are significant:
Instead of uncontrolled expansion under pressure caused by the culture movement, the tubes will expand to their limit and become fully rigid and hold their shape
This rigidity will a) keep the algae culture at the waterline and b) keep the tube fully stretched out along its axis which will keep the ends of the reactor (the manifolds) from coming together
The curvature in the tubes means that there will be more photosynthetically active surface area when combining all the tubes than if the tubes were one piece of plastic film

These tubes together form one photobioreactor. The tubes are directly connected to the manifolds via direct physical contact which could be using clamps or other restraints.

Since the tubes are made of very light plastic film, the tube section of the photobioreactor will float in water of salinity greater than or equal to the algae culture. The manifolds will be heavier than water which will require that they be supported through a flotation unit attached to it such as pontoons.

The tubes and the manifold might have a similar shape at least in one dimension and might be made from different material with even very different characteristics, e.g. flexible material vs. rigid material. The connection between the flexible and rigid part might be created by gluing together the two parts or by using clamps or any other connection.

The tubes of the photobioreactor and the manifolds might have a different life-time. The connection between them might be constructed in such a way that the part with the shorter life-time, e.g., the tubes, can be replaced easily.

In order to operate a number of photobioreactors together, being able to autonomously estimate the density of the algae culture in each photobioeactor is important. The present invention accomplishes this via a system of light emitting points which shine light through the algae culture and then sensors which collect the refracted light. Based on the refraction rate, the culture density of the specific algae organism is estimated.

The photosynthetically active area absorbs sufficient sunlight that if there is not a good heat dispersion mechanism, the temperature inside the photobioreactor reactor rises beyond what is optimal growing conditions. The present invention attaches a heat exchanger made of the same flexible material as the photobioreactor to the photosynthetically active area. This could be attached inside or outside the flexible area, in both the top or bottom sheet. By passing cool water through this heat exchanger, the temperature of the culture inside the reactor will decline.

The present invention provides methods of growing photosynthetic or mixotrophic organisms. According to the method, a suspension comprising the organism is introduced into one of the floating photobioreactor systems of the present invention. The photobioreactor is located in a surrounding water body. The suspension is exposed to light and brought into contact with a gas mixture comprising CO₂ and other nutrients.

The present invention also provides methods of producing biomass. According to this method, a suspension comprising the photosynthetic or mixotrophic organisms is introduced into one of the floating photobioreactor systems of the present invention. The photobioreactor is located in a surrounding water body. The organisms are grown in a suspension in the photobioreactor. The suspension is exposed to light and brought into contact with a gas mixture comprising CO₂ and other nutrients. The organisms produce biomass, which is then harvested. The biomass may be harvested by methods known in the art.

The present invention also provides methods of producing biofuel. According to this method, a suspension comprising the photosynthetic or mixotrophic organisms is introduced into one of the floating photobioreactor systems of the present invention. The photobioreactor is placed (operated in?) in a surrounding water body. The organisms are grown in a suspension in the photobioreactor. The suspension is exposed to light and brought into contact with a gas mixture comprising CO₂ and other nutrients. The organisms produce biomass, which is then harvested. Lipids, carbohydrates, proteins, vitamins, antioxidants, components from the photosynthetic or mixotrophic organism, and other components from the biomass are converted into biofuel. The conversion may be performed by methods known in the art.

The present invention also provides methods of producing a product selected from the group consisting of biochemicals, amino acids, fine chemicals, nutriceuticals, pharmaceuticals, energy products (ethanol, methane, hydrogen, fatty acids, fats and other lipids, highly energetic compound, propanol, butanol, gasoline-like fuel, diesel-like fuel, alkanes, alkenes, alcohols, organic acids, aromatic compounds), protein, feed for cattle or other species, fish feed, including feed for fish larvae and feed for other potential aquaculture uses, e.g., food for shrimps, crabs, oysters and their larvae, protein source for human nutrition and mineral rich food for human consumption. According to this method, a suspension comprising the photosynthetic or mixotrophic organisms is introduced into one of the floating photobioreactor systems of the present invention. The photobioreactor is operated in a surrounding water body. The organisms are grown in a suspension in the photobioreactor. The suspension is exposed to light and brought into contact with a gas mixture comprising CO₂ and other nutrients. The organisms produce biomass, which is then harvested. Lipids, carbohydrates, proteins, vitamins, antioxidants, components from the photosynthetic or mixotrophic organism, and other components from the biomass are converted into the desired product. The conversion may be performed by methods known in the art.

While particular materials, formulations, operational sequences, process parameters, and end products have been set forth to describe this invention, they are not intended to be limiting. Rather, it should be noted by those ordinarily skilled in the art that the written disclosures are exemplary only and that various other alternatives, adaptations, and modifications may be made within the scope of the present invention. Accordingly, the present invention is not limited to the specific embodiments illustrated herein.

What is claimed is:

1. A floating, horizontal tubular photobioreactor system to grow a photosynthetic or mixotrophic microorganism comprising:
a photosynthetically active area made of clear tubes; and
two manifolds connecting to the ends of the photosynthetically active area.

2. The floating photobioreactor system according to claim 1, wherein the tubes comprise a flexible material.

3. The floating photobioreactor system according to claim 1, wherein the manifolds comprise a rigid material.

4. The floating photobioreactor system according to any one of claims 1 to 3, wherein the photobioreactor is substantially horizontal.

5. The floating photobioreactor system according to any one of claims 1 to 4, further comprising a microorganism solution, which comprises a photosynthetic or mixotrophic microorganism and a growth medium.

6. The floating photobioreactor system according to claim 5, wherein the manifolds contain integrated means to mix, aerate and degas the microorganism solution.

7. The floating photobioreactor system according to claim 6 wherein the manifold contains an integrated means to mix the algae culture

8. The floating photobioreactor system according to claim 7 wherein the means to mix the algae culture is an axial flow pump or other positive displacement pump which can be driven internally or externally from the manifold.

9. The floating photobioreactor system according to claim 6, further comprising a means for introducing gas into the manifolds.

10. The floating photobioreactor system according to claim 9, wherein the means for introducing gas is a perforated hose or a porous stone

11. The floating photobioreactor system according to claim 10, further the location of the aeration introduction being behind the flow of the stirring mechanism

12. The floating photobioreactor system according to claim 6, wherein the manifolds contain an integrated means to degas the culture

13. The floating photobioreactor system according to claim 12, wherein the method to degas the culture is a system which separates the exhaust gas from the remaining algae culture using gravity

14. The floating photobioreactor system according any of the claims 1 to 7, wherein the manifolds are directly connected to the photosynthetically active area without a hose or tube in between.

15. The floating photobioreactor system according to any one of claims 1 to 12, wherein the tubes in the photosynthetically active area are connected to the manifolds via glue, clamps or the like.

16. The floating photobioreactor system according to any of claims 1 to 3, wherein the level of the microorganism solution inside the manifolds are substantially the same as that inside the photosynthetically active area.

17. The floating photobioreactor system according to any of claims 1-14, floating on a supporting water body.

18. The floating photobioreactor system according to any one of claims 1 to 8, wherein the manifolds are connected to a fixed object.

19. The floating photobioreactor system according to claim 18, wherein the fixed object is the ground of the water body, a wall or a surrounding floating structure which could itself be attached to the ground of the water body.

20. The floating photobioreactor system wherein an autonomous culture density estimating system is comprised of light emitting sources passing light through the culture and then capturing the refracted light at sensors which creates a feedback loop for adjusting the operation of the photobioreactor.

21. A method of growing a photosynthetic or mixotrophic organism comprising:
(a) introducing a suspension comprising the photosynthetic or mixotrophic organism and growth medium into the floating photobioreactor system of any one of claims 1-20, wherein the photobioreactor is located in a surrounding water body;
(b) exposing the suspension to light; and
(c) contacting the suspension with a gas mixture comprising CO₂.

22. A method of producing a biomass comprising:
(a) growing a photosynthetic or mixotrophic organism in a growth medium in the floating photobioreactor system of any of claims 1-21, wherein the photobioreactor is surrounded by a water body;
(b) harvesting the biomass.

23. A method of producing a biofuel comprising:
(a) growing a photosynthetic or mixotrophic organism in a growth medium in the floating photobioreactor system of any one of claims 1-22, wherein the photobioreactor is surrounded by a water body;
(b) harvesting the organism; and
(c) converting one or more selected from the group consisting of lipids, carbohydrates, proteins, vitamins, or antioxidants from the organism, and components of the organism into the biofuel.

24. A method for producing a product comprising:
(a) growing a photosynthetic or mixotrophic organism in a growth medium in the floating photobioreactor system of any one of claims 1-23, wherein the photobioreactor is surrounded by a water body;
(b) harvesting the organism; and
(c) converting one or more selected from the group consisting of lipids, carbohydrates, proteins, vitamins, or antioxidants from the organism and components of the organism into the product,
wherein the product is selected from the group consisting of biochemicals, amino acids, fine chemicals, nutriceuticals, pharmaceuticals, energy products, protein, feed for cattle or other species, fish feed, protein source for human nutrition and mineral rich food for human consumption.
